(19) United States
(12) Patent Application Publication
(10) Pub. No.: US 2024/0065867 A1
(43) Pub. Date: Feb. 29, 2024

(54) AORTIC PROSTHESIS DELIVERY DEVICE AND METHOD OF USE

(71) Applicant: Bolton Medical, Inc., Sunrise, FL (US)

(72) Inventor: Timothy Lostetter, Sunrise, FL (US)

(21) Appl. No.: 18/503,569

(22) Filed: Nov. 7, 2023

Related U.S. Application Data
(63) Continuation of application No. PCT/US2022/028079, filed on May 6, 2022.
(60) Provisional application No. 63/186,954, filed on May 11, 2021.

(51) Int. Cl. A61F 2/95

(52) U.S. Cl. CPC A61F 2/9517

(57) ABSTRACT

A hub is slidable along a straight track, and an introducer sheath is fixed to and extends distally from the hub. A proximal handle is longitudinally slidable along the straight track and defines a helical track that has a travel length along the longitudinal axis. The hub and the proximal handle are mated, whereby rotation of the handle about the hub causes longitudinal travel of the hub and retraction of the introducer sheath from a prosthesis radially constrained within the introducer sheath. The hub proximally slidable along with straight track, thereby enabling retraction of the introducer sheath from the prosthesis by at least one of travel of the hub along the helical track during rotation of the proximal handle about the longitudinal axis, and by proximal longitudinal movement of the proximal handle along the straight track.

### CROSS-REFERENCE TO RELATED APPLICATION

This application is a Continuation of International Application PCT/US22/28079, filed on May 6, 2022, which claims the benefit of U.S. Provisional Patent Application No. 63/186,954, filed May 11, 2021. The entire contents of each prior application are incorporated herein by reference.

### BACKGROUND OF THE INVENTION

Aortic pathologies, including aortic aneurysms, are often treated by open surgical reconstruction, or alternatively, by endovascular repair, which is a minimally invasive alternative to open surgical repair. Optimizing a successful outcome of endovascular repair, however, requires assessment of the anatomy of the patient. In the case of an arterial or, more specifically, a thoracic or abdominal aortic aneurysm, an appropriate prosthesis spanning the proximal and distal ends of the aneurysm ensures complete exclusion of the aneurysm sack by properly anchoring the prosthesis in the aorta to minimize endoleaks and movement of the prosthesis in the aorta. In view of the typically close quarters in which the surgeon is required to operate, devices ancillary to endovascular delivery of aortic prostheses can be cumbersome, particularly if they occupy significant amounts of space and are not easily manipulated during the often precise orientation of the prostheses required prior to release at the designated landing sites spanning the aneurysm.

Therefore, need exists for new and improved endovascular repair devices and methods to treat aortic pathologies, such as aortic aneurysms.

### SUMMARY OF THE INVENTION

The present invention relates to an aortic prosthesis delivery system and method of its use in treating and repairing of aortic vascular damage, such as vascular damage associated with aortic aneurysms, and regions of the aorta having arterial branches that supply blood to vital organs and tissue, such as thoracic aortic aneurysms, abdominal aortic aneurysms, and thoracoabdominal aortic aneurysms; and, more specifically, juxtarenal aortic aneurysms and short-neck abdominal aortic aneurysms that employ fenestrated endovascular aortic repair.

In one embodiment, an aortic prosthesis delivery system of the invention includes a distal handle, a straight track extending along a longitudinal axis between a proximal end in a distal end of his distal handle, a hub at the straight track and movable along the straight track, and an introducer sheath fixed to and extending distally from the hub, whereby the introducer sheath extends distally from the distal handle. A proximal handle proximal to the distal handle is longitudinally slidable along the straight track. The proximal handle defines a helical track that has a travel length along the longitudinal axis of the hub and is rotatable about the longitudinal axis. The hub and the proximal handle are mated, so that rotation of the proximal handle about the hub causes longitudinal travel of the hub along a length of the longitudinal axis and the straight track relative to the proximal handle, while the proximal handle remains fixed relative to the distal handle. The length of travel of the hub is limited by length of the helical track of the hub. The hub and introducer sheath are longitudinally movable along the straight track by at least one of application of direct longitudinal force to the proximal handle and rotation of the proximal handle about the longitudinal axis.

In another embodiment, the invention is directed to a method of delivering an aortic prosthesis, such as a stent graft, to a surgical site. In this embodiment, an aortic prosthesis that is radially constrained within a distal end of an introducer sheath through an artery to an aortic aneurysm of a patient, whereby a distal end of the introducer sheath is proximal to the aneurysm is delivered to an aortic aneurysm of a patient. A proximal handle, that is proximal to and abutting a distal handle is rotated, wherein the proximal handle is mated to a hub at a helical track defined by an interior surface of the proximal handle and the hub is fixed to a proximal end of the introducer sheath, is rotated to thereby cause the hub to travel proximately along a straight track extending within the proximal handle and distally from the distal handle that abuts the proximal handle, whereby the introducer sheath is at least partially retracted from the aortic prosthesis at the aortic aneurysm, and at least partially released from radial constraint by the introducer sheath.

This invention has many advantages. For example, the length of the handle of the delivery device is minimized by limiting the travel length of a hub from which an introducer sheath distally extends. More specifically, when the proximal handle and the distal handle are in an abutting relationship to each other, the travel length of the hub from which an introducer sheath distally extends, can be limited to the length of the proximal handle. Further, by defining a helical track along which the hub can travel within the proximal handle, rotation of the proximal handle about the longitudinal axis permits travel of the hub along the straight track within the proximal handle or, alternatively retraction of the proximal handle in a proximal direction along the straight track. Therefore, the hub can be caused to move in a proximal direction, thereby retracting introducer sheath from an aortic prosthesis contained within it without extending the length of the delivery device beyond the cumulative longitudinal length of the proximal and distal handles, so long as they are in abutting relation to each other. This can be critical during orientation of an aortic prosthesis at a surgical site and before complete release of the aortic prosthesis. Further, retraction of the introducer sheath during orientation of the prosthesis is made more accurate by rotation of the proximal handle, which applies leverage and, therefore, greater control over retraction of the introducer sheath. Simultaneously, or alternatively, the surgeon has the option of simply moving the proximal handle in a proximal direction toward the surgeon along the straight track, thereby further, and more directly retracting the hub along the straight track and the introducer sheath from the surgical site. Only when the proximal handle is directed proximally toward the surgeon along the straight track does the total longitudinal dimension of the delivery device increase longitudinally beyond the cumulative longitudinal dimension of the proximal and distal handles.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a prospective view of one embodiment of the aortic prosthesis delivery system of the invention before retraction of an introducer sheath to thereby release a prosthesis radially constrained by the introducer sheath.

FIG. 2 is a cross-section, in perspective, of the proximal and distal handle portions of the aortic prosthesis delivery system of the invention at the starting position shown in FIG. 1, prior to retraction of the introducer sheath from a prosthesis constrained by and within the introducer sheath.

FIG. 3 is a perspective view of the aortic prosthesis delivery system of the invention shown in FIG. 2 after rotating the proximal handle component to thereby retract the introducer sheath from the prosthesis.

FIG. 4 is a perspective view, in cross-section, of the embodiment shown in FIG. 3, following retraction of the introducer sheath from the prosthesis.

FIG. 5 is a perspective view, in cross section, of the proximal and distal handles of FIG. 2, following proximal travel of the hub along the straight track by application of direct longitudinal force to the proximal handle without rotation of the proximal handle.

FIG. 6 is a perspective view, in cross-section, of the aortic prosthesis delivery system of the invention shown in FIG. 1, following retraction of the introducer sheath by application of direct longitudinal force to the proximal handle without rotation of the proximal handle.

### DETAILED DESCRIPTION OF THE INVENTION

The features and other details of the invention, either as steps of the invention or as combinations of parts of an invention, will now be more particularly described and pointed out in the claims. It will be understood that the particular embodiments of the invention are shown by way of illustration and not as limitations of the invention. The principal features of this invention can be employed in various embodiments without departing from the scope of the invention. It will also be understood that the same number appear in a different drawings represents the same item.

The invention is generally directed to an aortic prosthesis delivery system and the method of its use for delivering vascular prostheses in treating and repairing vascular damage, such as vascular damage associated with an aortic aneurysm, including regions of the aorta having arterial branches that supply blood to vital organs and tissues, such as juxtarenal aortic aneurysms and short-neck abdominal aortic aneurysms.

A description of example embodiments follows.

When reference is made to a stent graft, also referred to herein as “prosthesis,” “stent graft prosthesis,” or “vascular prosthesis” to be delivered or implanted in a patient, the word “proximal” means that portion of the prosthesis or component of the prosthesis that is relatively close to the heart of the patient. “Distal” means that portion of the prosthesis or component of the prosthesis that is relatively far from the heart of the patient.

When, however, reference is made to a delivery system or component of a delivery system employed to deliver, or implant, a prosthesis, the word “proximal,” as employed to herein, means closer to the clinician using the delivery system. When reference is made to a delivery system or component of a delivery system, “distal,” as that term is employed herein, means, further away from the clinician using the delivery system.

For clarity, the word “proximate” means “close to,” as opposed to the meanings ascribed to “proximal” or “distal” described above with respect to either the prosthesis or delivery system.

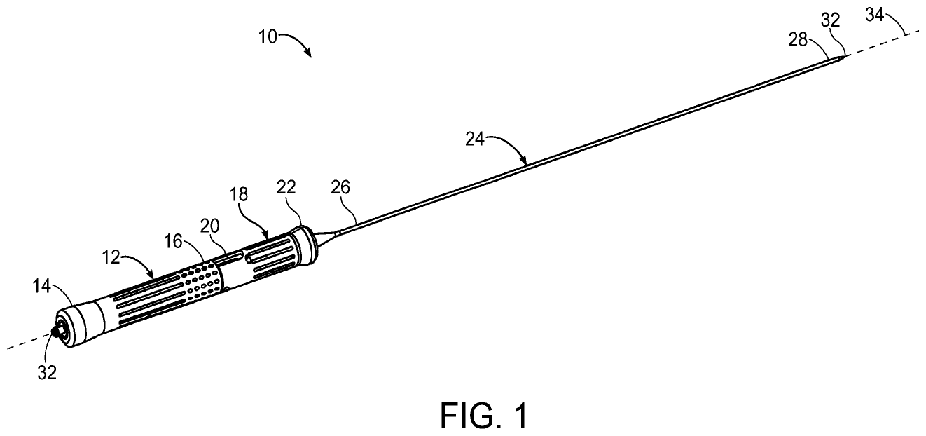

One embodiment of the aortic prosthesis delivery system of the invention is shown in FIG. 1. As shown therein, aortic prosthesis delivery system 10 includes proximal handle 12 having proximal end 14 and distal end 16, and distal handle 18 having proximal end 20 and distal end 22. Introducer sheath 24 has proximal end 26 and distal end 28, and extends distally from distal end 22 of distal handle 18, to which it is attached. A prosthesis, not shown, is radially constricted within distal end 28 of introducer sheath 24. Guidewire catheter 32 extends through proximal handle 12, distal handle 18, and introducer sheath 24.

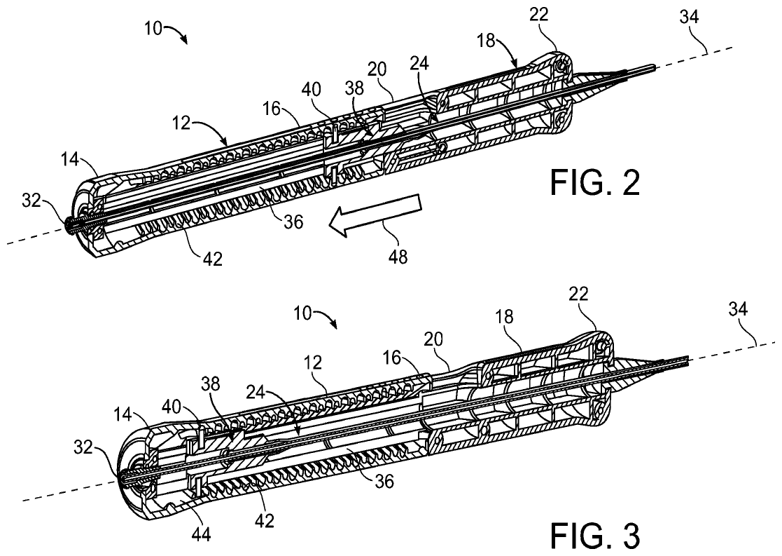

There are two methods by which introducer sheath 24 can be retracted from the prosthesis to expose and thereby release the prosthesis from radial constriction. FIG. 2 is a perspective view, in cross-section, of one embodiment of a proximal portion of aortic prosthesis delivery system 10 shown in FIG. 1 in a starting position, before deployment of the prosthesis. As shown in FIG. 2, proximal handle 12 and distal handle 18 are in abutting relation to each other, and the prosthesis, not shown, is radially constricted within distal end 28 of introducer sheath 24, but extends circumferentially about guidewire catheter 32.

As can be seen in FIG. 2, distal handle 18 has proximal end 20 and distal end 22. Proximal end 20 and distal end 22 define longitudinal axis 34. Straight track 36 extends proximally from the distal handle 12 and along longitudinal axis 34. Hub 38 is at straight track 36 and slidable along straight track 36. Rim 40 is at hub 38. Introducer sheath 24 is fixed to and extends distally from hub 38. The length of longitudinal travel of hub 38 along longitudinal axis 34 is limited by the length of straight track 36. Travel of hub 38 also is limited to travel along helical track 42 of proximal handle 12. Introducer sheath 24 extends distally from distal handle 18. Proximal handle 12 is proximal to distal handle 18 and is longitudinally slidable along straight track 36. Inside surface 44 of proximal handle 12 defines helical track 42 that has a longitudinal travel length along longitudinal axis 34. Proximal handle 12 is rotatable about longitudinal axis 34. Proximal handle 12 and hub 38 are mated, whereby rotation of proximal handle 12 about hub 38 causes longitudinal travel of hub 38 along a length of longitudinal axis 34 and straight track 36 relative to proximal handle 12 while proximal handle 12 remains longitudinally fixed relative to distal handle 18. As shown in FIG. 2, rim 40 of hub 38 engages helical track 42 of proximal handle 12 to thereby cause hub 38 to travel along straight track 36 as a consequence of rotation of proximal handle 12 about longitudinal axis 34.

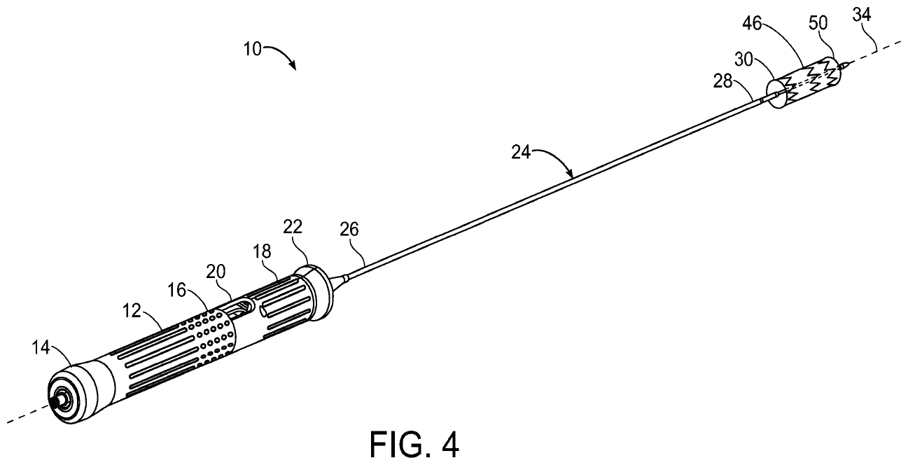

FIG. 3 is a perspective view, in cross-section, of proximal handle 12 and distal handle 18 of aortic prosthesis delivery system 10 shown in FIGS. 1 and 2, following retraction of introducer sheath 24 from prosthesis 46 (FIG. 4), having proximal end 30 and distal end 50, by rotation of proximal handle 12 (FIG. 3) about longitudinal axis 34 while proximal handle 12 is in abutting relation to distal handle 18. In one embodiment of a method of use of aortic prosthesis delivery system 10 of the invention, shown in the transition from FIG. 2 to FIG. 3, rotation of proximal handle 12 relative to hub 42 and straight track 36 causes rim 40 of hub 38 to travel along helical track 42 of proximal handle 12. As a consequence, depending on the direction of rotation of proximal handle 12, hub 38 travels along straight track 36 in a proximal direction. Introducer sheath 24 is fixed to hub 38 and, therefore, proximal travel of hub 38 along straight track 36 by rotation of proximal handle 12 causes retraction of introducer sheath 24 and consequent exposure of prosthesis 46, as appears in FIG. 4.

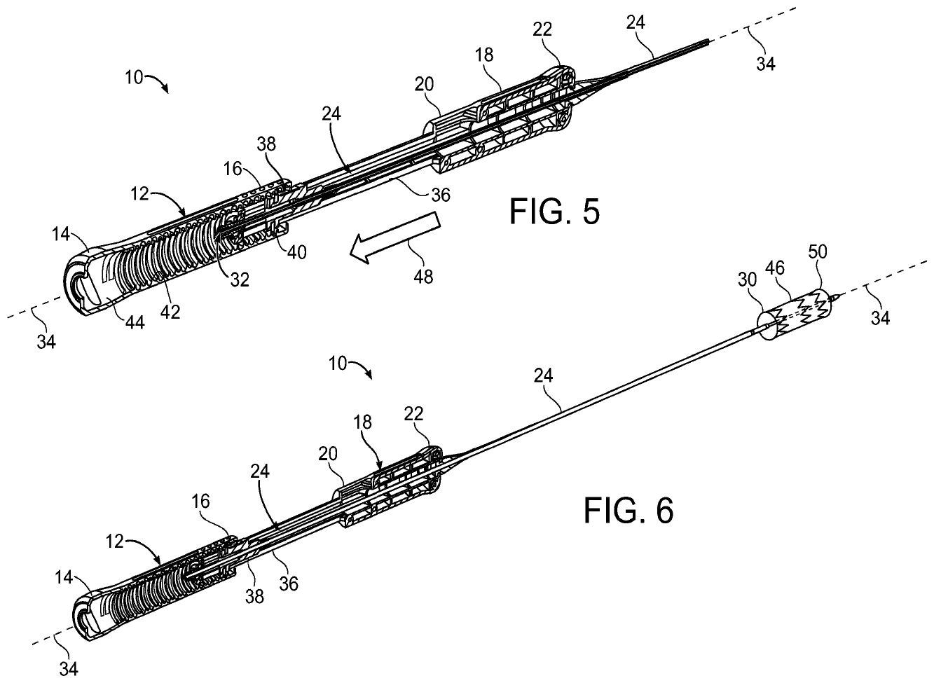

Alternatively, introducer sheath 24 can be retracted from prosthesis 46 by applying direct longitudinal force on proximal handle 12 in proximal direction 48 without rotation of proximal handle 12 about longitudinal axis 34. In this embodiment of a method of use of the aortic vascular delivery system 10 of the invention, hub 38 can be caused to travel proximally along straight track 36, thereby retracting introducer sheath 24 from prosthesis 46, by applying direct longitudinal force to proximal handle 12 in a proximal direction 48, toward the surgeon, along straight track 36. Specifically, as can be seen in the transition from FIG. 3 to FIG. 5, proximal handle 12 separates from distal handle 18, thereby distancing distal end 16 of proximal handle 12 from the proximal end 20 of distal handle 18. In this embodiment, longitudinal separation of proximal handle 12 from distal handle 18 causes retraction of introducer sheath 24 from prosthesis 46 and release of prosthesis 46 from radial constriction by introducer sheath 24, as can be seen in FIG. 6.

There are various embodiments of the method of use of the invention to implant a prosthesis, such as prosthesis 46, at a vascular site. Initially, proximal handle 12 and distal handle 18 can be manipulated to direct introducer sheath 24, which is radially constricting prosthesis 46 at distal end 28 of introducer sheath 24 to a surgical site spanning an aortic aneurysm. Referencing FIGS. 1-6, introducer sheath 24 can then be at least partially retracted from prosthesis 46, thereby at least partially releasing prosthesis 46 from radial constriction within the introducer sheath 24. In one embodiment, proximal handle 12 is rotated to thereby partially retract introducer sheath 24. Rotation of proximal handle 12 to retract introducer sheath 24 is leveraged by causing rim 40 of hub 38 to travel along helical track 42 defined by inside surface 44 proximal handle 12, thereby providing control over the position of introducer sheath 24 relative to prosthesis 46. Such control is particularly important during orientation of proximal end 50 of prosthesis 46 relative to an optimal landing site. Once the position of proximal end 50 of prosthesis 46 has been optimized and introducer sheath 24 has been sufficiently retracted to enable landing of proximal end 50 of prosthesis 46, retraction of introducer sheath 24 can be continued. Continued retraction of introducer sheath 24 can be conducted either by rotation of proximal handle 12, thereby causing continued travel of hub 38 along helical track 42 of proximal handle 12 or, alternatively, by directing proximal handle 12 in proximal direction 48 along straight track 36, thereby enabling a direct relation between the position of proximal handle 12 and the degree of retraction of introducer sheath 24 relative to prosthesis 46.

In another embodiment of a method of use of the aortic vascular delivery system 10 of the invention, introducer sheath 24 can be partially retracted by rotation of proximal handle 12 while it abuts distal handle 18, followed by further retraction of introducer sheath 24 by directly pulling proximal handle 12 in proximal direction 48. Further retraction of introducer sheath 24 can be completed, thereby fully exposing prosthesis 46, or only partially. When further retraction by directly pulling proximal handle 12 and, therefore, hub 38 along straight track 36, is partial, proximal handle 12 can then be rotated to bring proximal handle 12 back into abutting relation with distal handle 18, and then further rotated to continue retraction of introducer sheath 24 while it is in abutting relation with distal handle 18. In this way, leverage and control afforded by travel of rim 40 of hub 38 along helical track 42 of proximal handle 12 can be applied in intervals, and interrupted by more rapid retraction enabled by directly pulling proximal handle 12 in proximal direction 48 during the course of implantation of prosthesis 46. The overall length of the portion of aortic vascular delivery device 10 can thereby be minimized during implantation.

Following complete release of prosthesis at the surgical site, aortic prosthesis delivery system 10 can be retracted from the patient. Orientation of proximal end 50 of prosthesis 46 relative to the surgical site is particularly important where, for example, prosthesis 46 is fenestrated and must be rotationally oriented to align at least one fenestration defined by prosthesis 46 with an arterial branch blood vessel. The options for control over retraction of an introducer sheath 24 can also be advantageous when, for example, prosthesis 46 that is to be delivered is a branch prosthesis that must be directed through a fenestration of a previously implanted prosthesis, and directed into a branch blood vessel.

The relevant teachings of all patents, published applications and references cited herein are incorporated by reference in their entirety. The relevant teachings of U.S. Pat. Nos. 8,292,943; 7,763,063; 8,308,790; 8,070,790; 8,740,963; 8,007,605; 9,320,631; 8,062,349; 9,198,786; 8,062,345; 9,561,124; 9,173,755; 8,449,595; 8,636,788; 9,333,104; 9,408,734; 9,408,735; 8,500,792; 9,220,617; 9,364,314; 9,101,506; 8,998,970; 9,554,929; 9,439,751; 9,592,112; 9,655,712, 9,827,123, 9,877,857, 9,907,686; 10,105,248; 10,307,275; 10,524,893; 10,390,932; 10,213,291; 10,646,365; 10,390,932; 10,898,357; U.S. patent application Ser. Nos. 14/272,818; 15/478,424; 15/604,032; 16/507,304; 15/672,404; 16/414,292; PCT/US2017/025849; 16/379,423; 16/379,490; 16/379,354; 16/391,843; 16/391,995; 16/392,443; 16/414,208; 16/414,132; 16/433,654; 16/433,823; 16/513,559; and 17/200,213 are also incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. An aortic prosthesis delivery system, comprising:
a) a distal handle, the distal handle having a proximal end and a distal end, the proximal end and the distal end defining a longitudinal axis;
b) a straight track extending proximally from the distal handle and along the longitudinal axis;
c) a hub at the straight track and slidable along the straight track;
d) an introducer sheath fixed to and extending distally from the hub, whereby the introducer sheath extends distally from the distal handle;
e) a proximal handle proximal to the distal handle and longitudinally slidable along the straight track, wherein the proximal handle defines a helical track that has a longitudinal travel length along the longitudinal axis, the proximal handle being rotatable about the longitudinal axis, and wherein the hub and the proximal handle are mated, whereby rotation of the proximal handle about the hub causes longitudinal travel of the hub along a length of the longitudinal axis and the straight track relative to the proximal handle while the proximal handle remains fixed relative to the distal handle, the length of travel of the hub being limited by the travel length of the helical track of the hub, the hub and the introducer sheath being proximally movable along the straight track by at least one of rotation of direct longitudinal force on the proximal handle and rotation of the proximal handle about the longitudinal axis.

2. The aortic prosthesis delivery system of claim 1, wherein the hub includes a raised rim, whereby the raised rim of the hub follows the helical track of the proximal handle as the proximal handle is rotated about the longitudinal axis to thereby cause proximal movement of the hub and introducer sheath.

3. The aortic prosthesis delivery system of claim 2, wherein the rim of the hub extends radially outward from the hub and from the longitudinal axis, and wherein the helical track defines at least a portion of an interior space of the proximal handle.

4. The aortic prosthesis delivery system of claim 3, wherein the hub is confined to the interior space of the proximal handle.

5. The aortic prosthesis delivery system of claim 4, further including a guidewire catheter extending through the hub, the distal handle, the proximal handle, and the introducer sheath.

6. A method for treating an aortic aneurysm, comprising the steps of:
a) delivering an aortic prosthesis that is radially constrained within a distal end of an introducer sheath through an artery to an aortic aneurysm of a patient, whereby a distal end of the introducer sheath is proximal to the aneurysm;
b) rotating a proximal handle proximal to and abutting a distal handle, and that is mated to a hub at a helical track defined by an interior surface of the proximal handle, the hub being fixed to a proximal end of the introducer sheath, thereby causing the hub to travel proximately along a straight track extending within the proximal handle and distally from the distal handle that abuts the proximal handle, whereby the introducer sheath is at least partially retracted from the aortic prosthesis at the aortic aneurysm, and at least partially released radial constraint by the introducer sheath.

7. The method of claim 6, wherein the introducer sheath is partially retracted from the aortic prosthesis, and further including the step of applying proximal longitudinal force to the proximal handle, thereby causing the hub to slide proximally along the straight track and the introducer sheath to further retract from the stent graft.

8. The method of claim 7, wherein the aortic prosthesis defines a fenestration and the aneurysm is at a portion of an artery that includes an arterial branch with which the fenestration must be aligned, and further including the step of rotationally orienting the aortic prosthesis to align the fenestration with the arterial branch.

9. The method of claim 8, wherein the step of applying proximal longitudinal force to the proximal handle is conducted after aligning the fenestration of the aortic prosthesis with the arterial branch.
